(19) United States
(12) Patent Application Publication
(10) Pub. No.: US 2008/0305018 A1
(43) Pub. Date: Dec. 11, 2008

(54) Photosterilization Reactor

(75) Inventor: Dieter Wolfgang Blum, Aldergrove (CA)

Correspondence Address: ROBERT D. GUNDERMAN, JR.;PATENT TECHNOLOGIES, LLC, 150 LUCIUS GORDON DRIVE, SUITE 205, WEST HENRIETTA, NY, 14586, US

(73) Assignee: ALBONIA INNOVATIVE TECHNOLOGIES LTD., Vancouver (CA)

(21) Appl. No.: 12/136,027

(22) Filed: Jun. 9, 2008

Related U.S. Application Data
(60) Provisional application No. 60/943,301, filed on Jun. 11, 2007.

(51) Int. Cl. B01J 19/12

(52) U.S. Cl. 422/186.3

(57) ABSTRACT

A photosterilization reactor for sterilization of a liquid or a gas using ultraviolet light is disclosed that overcomes many of the shortcomings of current ultraviolet sterilization systems and improves the efficiencies of the sterilization process. The photosterilization reactor has an optically coupled flow path for maximizing the cross-sectional interaction between the ultraviolet radiation and the liquid or gas in the flow path, a reactor vessel for the efficient entrapment, reflection and use of the generated and unabsorbed ultraviolet radiation, and operational improvements to the ultraviolet light source, thermal and mechanical shielding of the ultraviolet light source, and electrical safety improvements. The photosterilization reactor may further contain a novel fluoroscopic pathogen detector that provides for real time monitoring of pathogens in the liquid or gas flow path internal to the reactor.

### CROSS-REFERENCE TO RELATED PATENT APPLICATIONS

This application claims the benefit of the filing date of U.S. Provisional Patent Application No. 60/943,301 filed on Jun. 11, 2007

### BACKGROUND OF THE INVENTION

1. Field of the Invention

This invention relates generally to an apparatus for sterilizing a liquid or a gas, and more particularly to a novel and improved apparatus that provides for mutagenic sterilization of a liquid or a gas using ultraviolet light.

2. Description of Related Art

The use of certain wavelengths of ultraviolet light, such as UV-C (240-280 nm.) at adequate dosing (energy and exposure time) destroys the Deoxyribonucleic Acid (DNA) structure of many micro-organisms and bacteria, causing mutagenic damage to many pathogens, effectively killing these micro-organisms and bacteria. The use of ultraviolet light is therefore very useful in disinfection of drinking water, fruit juices, milk, other liquids, gases, and some solids or composites such as fruits and vegetables.

The application of such mutagenic radiation to liquids such as water for example, typically involves the placement of an appropriate ultraviolet light source (usually low-pressure long-arc Hg lamps, which are the most efficient at converting electrical energy to useful radiation @65%, but which have a low intensity per unit volume) inside an ultraviolet transmissive sleeve, housing or other structure comprised of fused silica (quartz.) The sleeve transmits almost all of the radiation generated by the ultraviolet lamp into tile liquid which flows around and is in contact with the sleeve, but not into lamp envelope itself.

The use of the sleeve serves two purposes, the first is the thermal isolation of the lamp envelope from the liquid (lamp envelope temperature is critical to its optimum operation) as the optimum lamp envelope temperature may be quite far removed from the temperature of the liquid (the actual thermal isolation is provided more so by the small air gap between the sleeve and the lamp envelope, than the sleeve's thermal conductivity.)

The second purpose of the sleeve is for the containment of lamp materials and associated components, such as mercury, in the event of physical damage or rupture to the lamp.

Other prior art applications and systems involve the helical coiling of ultraviolet transmissive hollow tubes (comprised of quartz or UV transmissive polymers, i.e., FEP, Tetrafluorethylene-Perfluorpropylene, tradenames DAIKIN NEOFLON®, DUPONT TEFLON®, and HOECHST HOSTAFLON®.) around an ultraviolet lamp and flowing a liquid or gas through the tubing.

Still further prior art applications and systems coat or sheath an ultraviolet lamp with an ultraviolet transmissive polymer (i.e., FEP) and allow the liquid to contact and flow past the polymer sheath. The polymer sheath provides some thermal isolation of the lamp envelope from the liquid and provides a flexible containment sheath for the lamp materials and components in case of breakage.

These prior art systems suffer from a number of drawbacks, the major one being the inefficient coupling of the generated mutagenic radiation into the liquid or gas flow path, thereby lowering the cross-sectional interaction there between. Such inefficiencies result in, for example, incomplete sterilization, excessive power consumption, shortened component lifetimes, and the like.

It is therefore an object of the present invention to provide a photosterilization reactor for liquids or gasses that provides an optically coupled flow path that maximizes the energy exchange and cross-sectional interaction between the ultraviolet mutagenic radiation and the liquid or gas in the flow path.

It is another object of the present invention to provide a photosterilization reactor for liquids or gasses that entraps the generated and unabsorbed mutagenic radiation and therefore further maximizes the energy exchange and cross-sectional interaction between the ultraviolet mutagenic radiation and the liquid or gas in the flow path.

It is a further object of the present invention to provide a photosterilization reactor for liquids or gasses which utilizes less electrical input power for a given generated mutagenic radiation dose per volume than prior art systems.

It is a further object of the present invention to provide a photosterilization reactor for liquids or gasses that provides for the optimal operation of the ultraviolet light source.

It is a further object of the present invention to provide a photosterilization reactor for liquids or gasses that provides for the thermal and mechanical shielding of the ultraviolet light source.

It is a further object of the present invention to provide a photosterilization reactor for liquids or gasses that provides for electrical safety improvements when servicing the ultraviolet light source.

It is a further object of the present invention to provide a photosterilization reactor for liquids or gasses that provides for the direct fluoroscopic detection of pathogens in the liquid or gas flow path internal to the reactor.

### SUMMARY OF THE INVENTION

In accordance with the present invention, there is provided a photosterilization reactor that has an ultraviolet light source, a liquid or gas flow path, and a reactor.

In one embodiment of the photosterilization reactor, the liquid or gas flow path describes a helically coiled form within the main body of the reactor itself, in close proximity to the ultraviolet light source, as the main body of the reactor is highly transmissive to ultraviolet radiation and thereby acts as an efficient optical coupler for the ultraviolet radiation from the ultraviolet light source into the flow path. The exterior surface of the reactor is coated with an ultraviolet radiation reflecting material, thereby constraining the generated ultraviolet radiation within the main body of the reactor.

In an alternative embodiment of the photosterilization reactor, there are provided means for recirculating and thereby re-sterilizing a portion of liquid or gas from a storage chamber or the like which is fed by the output of the reactor.

In a further embodiment of the photosterilization reactor, there are provided means internal to the reactor for sampling and holding or otherwise containing a small portion of the incoming liquid or gas stream at or from the input of the reactor, exposing the small sample portion to a multiplicity of organic fluoroscopic excitation wavelengths, said wavelengths being generated in situ at the wall of the sampling chamber within the reactor by means of a selected group of phosphors capable of down-converting a portion of the ultraviolet radiation from the ultraviolet light source, thereby effecting the fluorescence excitation of some of the pathogens that may be contaminating the input liquid or gas, and further there are provided means for detecting the fluorescence from said pathogens thereby providing for the direct fluoroscopic detection of pathogens within the reactor itself. In addition, the process of holding the small sample for a suitable time provides for an incubation period that may allow for the multiplication of some of the pathogens that may be contaminating the input liquid or gas, further improving the accuracy of this novel pathogenic detection system.

In a further alternative embodiment of the photosterilization reactor, there are provided means for embedding solid-state radiation detectors within the reactor body itself for detecting fluorescence radiation from pathogens if present in the liquid or gas sample.

In a further alternative embodiment of the photosterilization reactor, there are provided means for including or otherwise embedding a photomultiplier radiation detector assembly within a vacuum inclusion formed within the reactor body itself for detecting fluorescence radiation from pathogens if present in the liquid or gas sample.

In another alternative embodiment of the photosterilization reactor, there are provided means for exposing one or more sections of the liquid or gas sterilization flow path to a multiplicity of organic fluoroscopic excitation wavelengths, the wavelengths being generated in situ at the wall(s) of the liquid or gas sterilization flow path within the reactor by means of a selected group of phosphors capable of down-converting a portion of the ultraviolet radiation from the ultraviolet light source, thereby effecting the fluorescence excitation of some of the pathogens that may be contaminating the input liquid or gas, and also if desired, the output liquid or gas, and further there is provided a means for detecting fluorescence from pathogens thereby providing for the direct fluoroscopic detection of pathogens within the reactor itself.

In some of the preferred embodiments summarized above, the direct fluoroscopic detection of pathogens within the reactor itself allows for the closed-loop control of energization of the ultraviolet light source, as well as the liquid or gas flow rate and feedrate through the reactor, thereby allowing for precise control of the dwell time, exposure time and overall radiation dose provided to the liquid or gas flowing through the reactor.

Modifications and alternatives to the various embodiments of the present invention may be made without departing from the spirit and broad scope of the invention as described herein.

### BRIEF DESCRIPTION OF THE DRAWINGS

The invention will be described by reference to the following drawings, in which like numerals refer to like elements, and in which:

FIG. 1 is a vertical cross-sectional view of a photosterilization reactor in accordance with one embodiment of the present invention;

FIG. 2 is a vertical cross-sectional view of a photosterilization reactor in accordance with another embodiment of the present invention;

FIG. 3 is a vertical cross-sectional view of a photosterilization reactor showing a pathogen detection system in accordance with a further embodiment of the present invention;

FIG. 4 is a vertical cross-sectional view of a photosterilization reactor showing additional pathogen detection in accordance with a further embodiment of the present invention;

FIG. 5 is a close up cutaway view of a photosterilization reactor with pathogen detection.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by this specification and claims and the attached drawings.

### DESCRIPTION OF THE PREFERRED EMBODIMENTS

For a general understanding of the present invention, reference is made to the drawings. In the drawings, like reference numerals have been used throughout to designate identical elements.

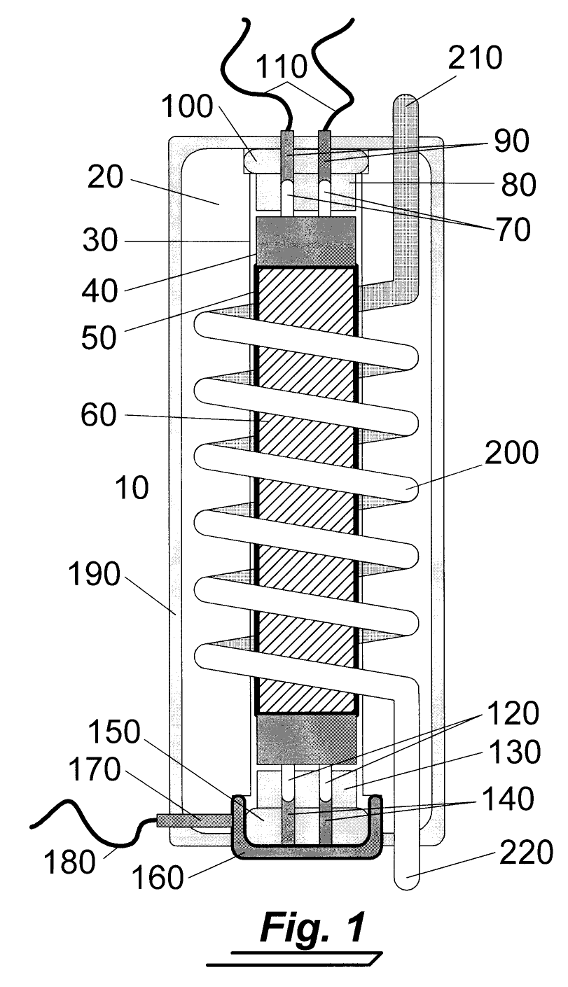

FIG. 1 shows a photosterilization reactor 10. The photosterilization reactor 10 comprises a reactor body 20 made of suitable ultraviolet (UV-C) light transmissive material (minimum absorbance/maximal transmissivity in the region of 240-260 nm) such as fused silica (quartz), or preferentially of a UV transmissive thermoplastic (e.g., CORNING COSTAR®, ZEON ZEONEX® or ZEONOR®, TORAY RAYTELA®, CYP or the like), which are extensively used in the manufacture of UV transmissive wells and cuvettes for fluoroscopy applications.

Axially disposed in the center of the reactor body 20 is a tubular channel 30 for holding and constraining a suitable ultraviolet light source, here shown to be a low-pressure long-arc mercury vapor (Hg) lamp 40. Other lamps that provide ultraviolet emissions in the desired wavelength may also be used. The UV lamp 40 fits snugly into the channel 30 with a minimal airgap between the wall of the channel 30 and the outside of the envelope (quartz) 50 of the UV lamp 40. The envelope 50 of said lamp UV 40, in some embodiments of the present invention, will be in contact with the wall of channel 30 at many places, but due to the low thermal conductivity of the reactor body 20, the UV lamp envelope will operate at an optimal temperature.

The UV lamp 40, serves to efficiently generate short-wave ultraviolet radiation (UV-C), predominately at about 253.7 nm (i.e., Hg resonance radiation) which effects mutagenic and disruptive damage to microorganisms. At a wavelength of 2,537 Angstroms (254 nm) UV will break the molecular bonds within the DNA of microorganisms, producing thymine dimers in the DNA, thereby destroying them, rendering them harmless or prohibiting growth and reproduction.

The UV lamp 40 is electrically energized as follows (electronic controller not shown for clarity). At the sealed, upper end of reactor 20, the UV lamp 40 can be seen to have the upper two electrical connections (bi-pin) 70 for its upper filament/electrode (hot-cathode lamp is shown, as it is far more efficient than cold-cathode, as is well known in the art) in contact with two hollow mating electrical contacts 90 fixed rigidly in the reactor body 20 and which pass through an electrical insulator 80.

Said insulator 80 and the upper end of UV lamp 40 (which is in physical contact therewith) are held via an elastomeric bushing 100. Said contacts 90, insulator 80 and bushing 100 are all axially disposed within said tubular channel 30 as shown. Wires 110 lead from the contacts 90 to an electronic lamp controller (not shown for clarity).

The wires 110 and the external portions of contacts 90 are electrically insulated (not shown) so that all of the electrically active elements at this end of the UV lamp 40 are physically inaccessible, providing a great measure of safety, since during startup of the UV lamp 40 there may be potentials on the order of hundreds of volts present (depending on the length of UV lamp 40, this could be as high as 800 or more volts) at this upper end of the UV lamp 40, with respect to the lower end of UV lamp 40 and earth ground. During normal operation of the arc within UV lamp 40, the potential difference between the upper end thereof and the lower end (and earth ground) will normally lie between 70 and 200 volts (again, depending on the length of UV lamp 40.)

The only physically accessible electrically active elements are seen to be located at the lower end of UV lamp 40, and as shown, the lower two electrical connections (bi-pin) 120 for the lower filament/electrode is in contact with two hollow mating electrical contacts 140 that pass through an electrical insulator 130. The insulator 130 is in physical contact with the lower end of UV lamp 40 and so both are held by elastomeric bushing 150, which sits within a metallic/conductive retaining cap 160 (retention means not shown for clarity) that inserts into the reactor body 20. The contacts 140, insulator 130, bushing 150 and metallic/conductive retaining cap 160 are all axially disposed within said tubular channel 30 as shown.

Said electrical contacts 140 are in physical and electrical contact with the metallic/conductive retaining cap 160 and can be seen to be electrically short-circuited to each other and the end cap 160. An electrically conductive contact 170 is fixed rigidly within the reactor body 20 and serves to provide an electrical contact with retaining cap 160 when in place and thence to the two lower lamp contacts 120. A preferably insulated wire 180 leads from the contact 170 to an electronic lamp controller (not shown for clarity).

Although this lower electrical assembly is physically accessible (due to the exposed metallic/conductive retaining cap 160), there are no safety concerns since this lower electrical assembly (i.e., all components thereof) are at a potential of 0 volts with respect to earth ground, thereby not presenting an electrical safety hazard.

Disposed in a concentric helical fashion about the tubular channel 30 (housing the lamp) within the reactor body 20 is a flow path or channel 200 for the flow of fluid/liquid or gas and exposure thereof to the mutagenic ultraviolet radiation from the UV lamp 40 as coupled through the reactor body 20 itself to reach the channel 200 with very little attenuation.

The flow path 200 has an upper flow inlet 210 and a lower flow outlet for the admittance and exhaust of the fluid/liquid or gas to be irradiated with the reactor assembly 10. The terms “upper” and “lower” as used above, are not meant to restrict the operational orientation of the reactor, but are merely for descriptive convenience.

To further enhance operation, increase the efficiency of the irradiation process and to provide safety and protection from the harmful UV-C radiation to the outside of the reactor body 20, the exterior surface of the reactor body 20 is coated with a UV reflective layer 190 (such as Aluminum Oxide or other reflective material that may be applied as a thick-film or a thin-film, through vacuum deposition or similar process). The reflective layer 190 serves to contain or entrap the unabsorbed radiation within the reactor body 20 and ensures maximal interaction between the generated radiation and the fluid/liquid or gas within the flow path/channel 200.

The reactor body 20 may be molded or cast in two or more pieces, which are then bonded together to form a single unitary piece (the reactor body 20), and further coated with a reflective layer 190.

The tubular channel 30, along with the retaining cap 160, further serves to act as a containment chamber in the event that the UV lamp 40 ruptures or otherwise breaks, leaking contaminants such as mercury that could cause toxic contamination if the mercury were to come into contact with the fluid, liquid or gas in said flow path 200. As can be seen, there is no potential for such cross-contamination to occur within the reactor body 20 as shown and described here (provided there is no leak within the bond seam between body surfaces).

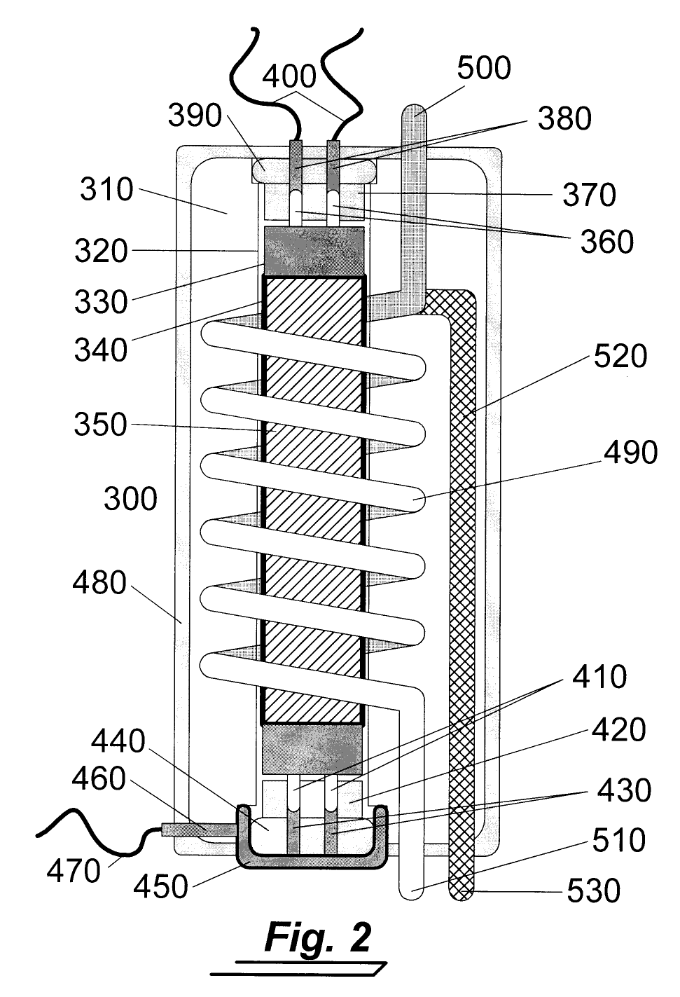

Now referring to FIG. 2, another embodiment of the photosterilization reactor 300 of the present invention is depicted. It is substantially the same as that described in FIG. 1, but with the addition of an ancillary flow channel 520, which couples into the main flow path 490 (200 in FIG. 1) at the inlet section and which serves to provide a recirculation flow path from lower recirculation inlet 530 to the main flow path 490. This is useful when the reactor is to be used as part of a treatment system containing a holding tank for sterilized fluid/liquid or gas and where it is expedient to recirculate a portion of the contents of said holding tank for re-sterilization.

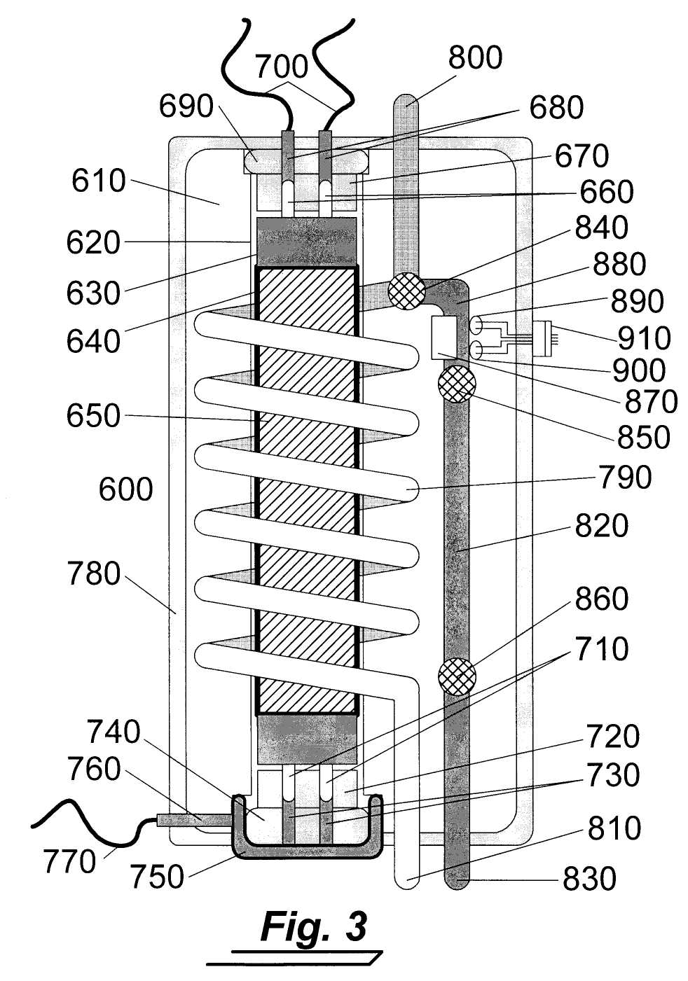

Now shown in FIG. 3 is another embodiment of the photosterilization reactor 600 of the present invention. It is substantially the same as that described in FIG. 2, but with the addition of a three-way valve 840 and two (2) single-acting valves 850 and 860; thereby portioning the ancillary flow path 820 into three distinct portions or sections. In some embodiments of the present invention, these valves can be integral to the reactor body. A compound phosphor layer 870 is placed on a portion of the exterior of the wall by forming a small thin void adjacent to the ancillary flow channel and inserting the phosphors therein of ancillary flow section/portion 880, for the down-conversion of UV-C radiation from the UV lamp 630. A photodetector array assembly 890, 900 and its external connections 910 are also depicted.

In this embodiment, the three-way valve 840 can block flow from inlet 800 to the main flow path 790 (sterilization/treatment path) and the ancillary path portion 880 simultaneously, or the valve 840 can direct all of the input flow from inlet 800 to the main flow path 790, or said valve 840 can direct the input flow from inlet 800 to the ancillary flow path 880.

With the topology described, the reactor can operate in a number of modes as follows:

MODE 1: All of the input flow from inlet 800 is directed through the main flow path 790 and exhausts out of output 830 with the UV lamp 630 not energized. In this passive flow-through mode there is no mutagenic radiation present and hence no sterilization takes place;

MODE 2: There is no flow from inlet 800 to/through the main flow path 790, but the valve 840 directs a flow from inlet 800 to ancillary flow channel 880, with valve 850 being closed, hence filling that portion between valve 840 and valve 850 with a fluid/liquid/gas sample to be analyzed. Valve 840 is then closed, trapping the sample in that portion between valves 840 and 850. Said UV lamp 630 is then pulsed (or modulated) in order to produce sufficient stimulating radiation to the compound phosphor 870 layer/patch, in order that said phosphor layer/patch 870 can down-convert said stimulating radiation via the emission of multiple less energetic wavelengths derived from the UV-C excitation energy.

In this analysis-only mode one can perform the direct fluoroscopic detection of pathogens within the sample via the use of photodetector assembly 890, 900, 910, or via the use of a photomultiplier assembly. This can be done after a time interval if desired, in order to allow pathogens to multiply. At the completion of the analysis valve 850 is opened, allowing the sample to enter into the channel space between valve 850 and valve 860 (which is kept closed.) Then valve 840 is briefly opened to allow a small amount of input flow from inlet 800 to “wash through” channel portion 880 between valve 840 and valve 850, thereby flushing the “sample” space, this small amount of input flow also ending up in the channel space between valve 850 and valve 860. Then with valve 840 closed, valve 850 open (or closed) and valve 860 closed, the UV lamp 630 is fully energized in order to sterilize the entirety of ancillary channel 820, including portion 880.

At the conclusion of a dose sufficient irradiation period, valve 860 is opened, dumping the contents (if liquid) of ancillary channel 820 out of exhaust outlet 830 into a suitable evaporation pan or the like, leaving the water to evaporate and leaving dead and dying pathogens thereon (no longer harmful.) If the working fluid is gas, a pressurized flush would need to be utilized, perhaps with an inert gas;

MODE 3: In this mode, modes 1 and 2 are combined/paralleled with the UV lamp 630 fully energized or energized at the power level required vs. flow rate, leading to simultaneous sterilization and analysis and hence a form of closed-loop operation for the reactor.

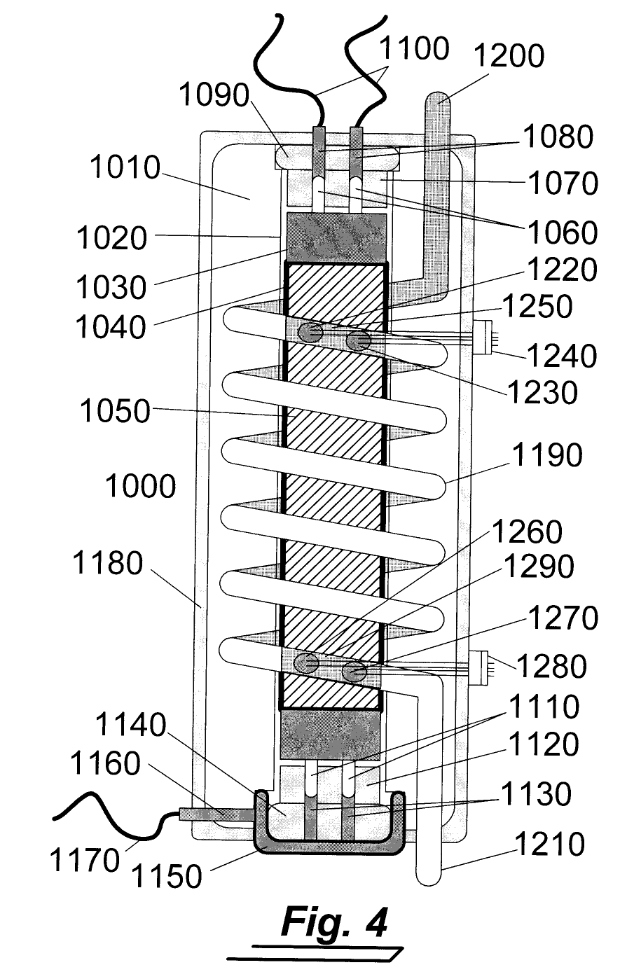

Referring now to FIG. 4, a further embodiment of the photosterilization reactor 1000 of the present invention is shown. It is substantially the same as that described in FIG. 1, but with the addition of an upper analysis/sensing section comprising a compound phosphor strip 1250 and photodetector assembly 1220, 1230 and 1240, and a lower analysis/sensing section having a compound phosphor strip 1290 and photodetector assembly 1260, 1270 and 1280, for the direct fluoroscopic pathogen detection at either the inlet/input side of the reactor, the exhaust/outlet side of the reactor, or both. Again, with the UV lamp 1030 fully energized or energized at the power level required for a given flow rate, this leads to simultaneous sterilization and analysis and hence also provides a form of closed-loop operation for the reactor.

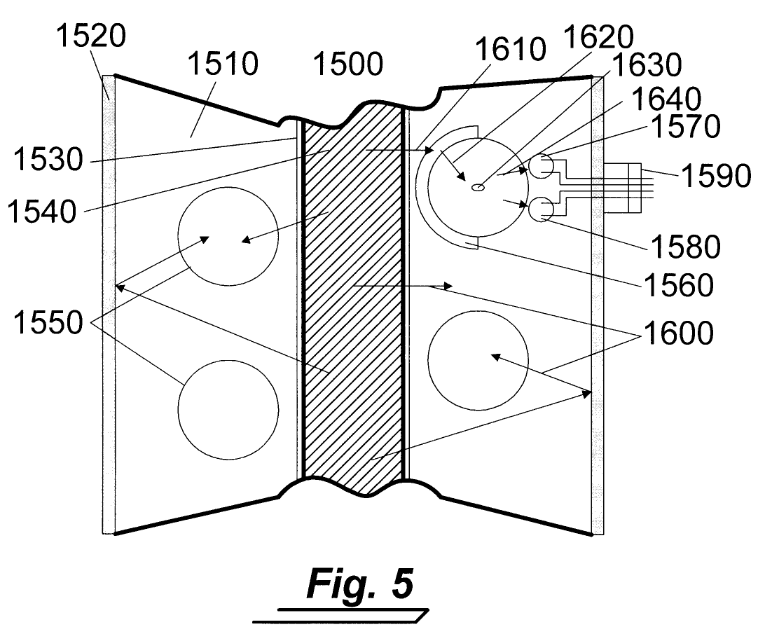

Referring lastly to FIG. 5, a close up cutaway view of a photosterilization reactor 1500 with pathogen detection is shown.

The reactor 1500 comprises a reactor body 1510 having a UV reflective coating 1520 upon its exterior surface(s) and containing a UV lamp 1540 within tubular channel 1530. Also shown in cross-section are portions of the helical flow path 1550, compound phosphor layer 1560, photodetector assembly 1570, 1580 and 1590. As can be clearly seen in FIG. 5, the UV lamp 1540 generates mutagenic UV-C radiation 1600, some of which radiation may directly impinge upon pathogens contained within the flow path 1550 and some of which radiation may miss the flow path 1550 altogether, only to be reflected back into the reactor body due to reflective layer 1520, with only minor attenuation and hence will eventually intersect and interact with the flow path 1550. As can be seen from FIG. 5, some of the UV-C radiation 1610 will impinge upon the compound phosphor patch/layer 1560, the phosphor layer 1560 being compounded to provide for the emission of desired stimulating wavelengths 1620 for the direct fluoroscopy detection of pathogens 1630 via the emission of further new and detectable wavelengths 1640.

In some embodiments of the present invention, the insides of the flow channels (main and ancillary) may be coated with Fluorinated Ethylene Propylene (FEP) to further increase slipperiness and inertness to further curtail the attachment of microorganisms.

The shape of the reactor is not meant to be limited to that shown in the drawings; other shapes and form factors can be used without departing from the scope of the present invention. There can also be one or multiple fluid paths. Shown in the drawings are helical/spiral paths, but other shapes and geometric configurations can be used without departing from the scope of the present invention. In some embodiments of the present invention, faces of the reactor block can be “dimpled” to increase scattering effects.

Although some features of the present invention are shown in certain drawings and not others, this is for convenience and basic teaching only, as some features may be combined with any or all of the other features without departing from the spirit and broad scope of the present invention.

It is, therefore, apparent that there has been provided, in accordance with the various objects of the present invention, a photosterilization reactor for sterilizing a liquid or a gas. While the various objects of this invention have been described in conjunction with preferred embodiments thereof, it is evident that many alternatives, modifications, and variations will be apparent to those skilled in the art. Accordingly, it is intended to embrace all such alternatives, modifications and variations that fall within the spirit and broad scope of the present invention as described by this specification and claims and the attached drawings.

What is claimed is:

1. A photosterilization reactor comprising:
a source of ultraviolet light confined by an ultraviolet light transmissive tubular channel;
an ultraviolet light transmissive flow path having an inlet and an outlet where the flow path is disposed in a concentric helical shape about the tubular channel; and
a reactor body containing the source of ultraviolet light, the tubular channel and the ultraviolet light transmissive flow path where the inlet of the flow path enters the reactor body and the outlet of the flow path exits the reactor body.

2. The photosterilization reactor as recited in claim 1, wherein the reactor body is made from fused silica quartz.

3. The photosterilization reactor as recited in claim 1, wherein the reactor body is made from an ultraviolet light transmissive thermoplastic.

4. The photosterilization reactor as recited in claim 1, wherein the source of ultraviolet light is a low pressure long arc mercury vapor lamp.

5. The photosterilization reactor as recited in claim 1, wherein the reactor body is coated with an ultraviolet light reflective layer.

6. The photosterilization reactor as recited in claim 1, further comprising an ancillary flow channel for recirculation and resterilization of a fluid source.

7. The photosterilization reactor as recited in claim 1, further comprising a fluorinated ethylene propylene coating on the inside of the flow path.

8. A photosterilization reactor comprising:
a source of ultraviolet light confined by an ultraviolet light transmissive tubular channel;
an ultraviolet light transmissive flow path having an inlet and an outlet where the flow path is disposed in a concentric helical shape about the tubular channel;
a reactor body containing the source of ultraviolet light, the tubular channel and the ultraviolet light transmissive flow path where the inlet of the flow path enters the reactor body and the outlet of the flow path exits the reactor body; and
an ancillary flow channel having a compound phosphor layer and a photodetector array for the detection of pathogens in fluid being processed by the photosterilization reactor.

9. The photosterilization reactor as recited in claim 8, wherein the reactor body is made from fused silica quartz.

10. The photosterilization reactor as recited in claim 8, wherein the reactor body is made from an ultraviolet light transmissive thermoplastic.

11. The photosterilization reactor as recited in claim 8, wherein the source of ultraviolet light is a low pressure long arc mercury vapor lamp.

12. The photosterilization reactor as recited in claim 8, wherein the reactor body is coated with an ultraviolet light reflective layer.

13. The photosterilization reactor as recited in claim 8, further comprising an ancillary flow channel for recirculation and resterilization of a fluid source.

14. The photosterilization reactor as recited in claim 8, further comprising a fluorinated ethylene propylene coating on the inside of the flow path.

15. A photosterilization reactor comprising:
a source of ultraviolet light confined by an ultraviolet light transmissive tubular channel;
an ultraviolet light transmissive flow path having an inlet and an outlet where the flow path is disposed in a concentric helical shape about the tubular channel;
a reactor body containing the source of ultraviolet light, the tubular channel and the ultraviolet light transmissive flow path where the inlet of the flow path enters the reactor body and the outlet of the flow path exits the reactor body; and
a compound phosphor strip and a photodetector array placed in optical communication with the outlet of the flow path for the detection of pathogens in fluid being processed by the photosterilization reactor.

16. The photosterilization reactor of claim 15 further comprising a compound phosphor strip and a photodetector array placed in optical communication with the inlet of the flow path for the detection of pathogens in fluid entering the photosterilization reactor.

17. The photosterilization reactor as recited in claim 15, wherein the reactor body is made from fused silica quartz.

18. The photosterilization reactor as recited in claim 15, wherein the reactor body is made from an ultraviolet light transmissive thermoplastic.

19. The photosterilization reactor as recited in claim 15, wherein the source of ultraviolet light is a low pressure long arc mercury vapor lamp.

20. The photosterilization reactor as recited in claim 15, wherein the reactor body is coated with an ultraviolet light reflective layer.

21. The photosterilization reactor as recited in claim 15, further comprising an ancillary flow channel for recirculation and resterilization of a fluid source.

22. The photosterilization reactor as recited in claim 15, further comprising a fluorinated ethylene propylene coating on the inside of the flow path.
